(19) United States
(12) Patent Application Publication
(10) Pub. No.: US 2005/0249676 A1
(43) Pub. Date: Nov. 10, 2005

(54) Pullulan capsules

(76) Inventors: Robert Scott, Chatton (GB); Dominique Cade, Colmar (FR); Xiongwei He, Andolsheim (FR); Ewart Thomas Cole, Hofstetten (CH)

Correspondence Address: PFIZER, INC., 201 TABOR ROAD, MORRIS PLAINS, NJ, 07950, US

(21) Appl. No.: 11/121,226

(22) Filed: May 3, 2005

(30) Foreign Application Priority Data
May 4, 2004 (EP) 04 291 151,1

(51) Int. Cl. A61L 9/04; A61K 9/14; A61K 31/715

(52) U.S. Cl. 424/46; 514/54

(57) ABSTRACT

The invention is a hard capsule comprising pullulan in an amount of 85% to 90% by weight, potassium chloride in an amount of 1.0% to 1.5% by weight, carrageenan in an amount of 0.1% to 0.4% by weight, one or more surfactants in an amount of 0.1% to 0.2% by weight and water in an amount of 10% to 15% by weight. Additionally the invention is related to new uses of pullulan containing containers.

### FIELD OF THE INVENTION

The invention concerns pullulan compositions for the use in pharmaceutical, veterinary, food, cosmetic or other products like films for packaging seeds, Micro-chips or for wrapping food, aspics or jellies, preferably for predosed formulations like soft or hard capsules. Furthermore, the present invention relates to predosed formulations for intratracheobronchial administration, predosed formulations able to dissolve in cold water and/or to dissolve without leaving residues behind.

### BACKGROUND OF THE INVENTION

Conventional hard capsules are made with gelatin by dip molding process. The dip molding process is based on the setting ability of hot gelatin solutions by cooling. For the industrial manufacture of pharmaceutical capsules gelatin is most preferred for its gelling, film forming and surface active properties. The manufacture of hard gelatin capsules by dip molding process exploits fully its gelling and film forming abilities. A typical dip molding process comprises the steps of dipping mould pins into a hot solution of gelatin, removing the pins from the gelatin solution, allowing the gelatin solution attached on pins to set by cooling, drying and stripping the so-formed shells from the pins. The setting of the solution on the mould pins after dipping is the critical step to obtain a uniform thickness of the capsule shell.

On a totally automatic industrial hard gelatin capsule machine, the process consists to dip mould pins into hot gelatin solution, to remove the pins from the solution, to turn the pins from downside to upside, to dry the gelatin solution (gel) attached on the pins, to strip the capsule shell and finally to cut and pre-joint the cap and body. The immediate setting of the gelatin solution on the dip pins after dipping is the key step in the process. Otherwise, the gelatin solution would flow down, leading to a very low top thickness, and no capsule of quality could be produced.

Gelatin-made hard medicinal capsules prepared in this manner, however, have several problems and disadvantages. For example, cross-linking phenomena, occurring in hard gelatin capsules, can cause considerable changes in the dissolution profiles of drugs. Incomplete in capsule shell dissolution and subsequent drug release problems is related to the fact that a variety of reagents are capable of interacting covalently with gelatin, e.g aldehydes, in particular formaldehyde.

Furtheron gelatin is liable to the attack of microorganisms so that the quality of gelatin-made capsule is unavoidably degraded in the lapse of time.

Attempts have been made to manufacture capsules with materials other than gelatin, notably with modified cellulose. Successful industrial examples are the capsules made of hydroxypropyl methylcellulose (HPMC).

Pullulan is a natural, viscous, water-soluble polysaccharide extracellularly produced e.g. by growing certain yeasts on starch syrups. It can be produced through a fermentation process. It has good film forming properties and a particularly low oxygen permeability and a moisture content at 50% RH of about 12%. Its existence was reported for the first time in 1938. Hayashibara Company started the commercial production in 1976.

There are numerous patents about the use of pullulan in molded articles, edible films, and coatings.

U.S. Pat. No. 4,623,394 describes a molded article which exhibits a controlled rate of disintegration under hydrous conditions. The composition of the molded article consists essentially of a combination of pullulan and a heteromannan, the amount of heteromannan being, based on the dry solids, 1 to 100% of the pullulan.

JP5-65222-A describes a soft capsule, capable of stabilizing a readily oxidizable substance enclosed therein, exhibiting easy solubility, and being able to withstand a punching production method. The soft capsule is obtained by blending a capsule film substrate such as gelatin, agar, or carrageenan with pullulan.

U.S. Pat. No. 3,784,390-A, corresponding to FR 2,147,112 and GB 1,374,199, discloses that certain mixtures of pullulan with at least one member of the group consisting of amylose, polyvinyl alcohol, and gelatin can be shaped by compression molding or extrusion at elevated temperatures or by evaporation of water from its aqueous solutions to form shaped bodies, such as films or coatings. To retain the valuable properties of pullulan to an important extent the mixture should not contain more than 120 percent amylose, 100 percent polyvinyl alcohol, and/or 150 percent gelatin based on the weight of the pullulan in the mixture.

U.S. Pat. No. 4,562,020, discloses a continuous process for producing a self-supporting glucan film, comprising casting an aqueous glucan solution on the surface of a corona-treated endless heat-resistant plastic belt, drying the glucan solution thereon while heating and releasing the resultant self-supporting glucan film. Suitable glucans are those which substantially consist of repeating maltotriose units, such as pullulan or elsinan.

JP-60084215-A2 discloses a film coating composition for a solid pharmaceutical having improved adhesive properties on the solid agent. The film is obtained by incorporating pullulan with a film coating base material such as methylcellulose.

JP-2000205-A2 discloses a perfume-containing coating for a soft capsule. The coating is obtained by adding a polyhydric alcohol to a pullulan solution containing an oily perfume and a surfactant such as a sugar ester having a high HLB.

U.S. Pat. No. 2,949,397 describes a method of making a mineral filled paper which comprises the step of coating finely divided mineral filler particles with an aqueous colloidal dispersion of plant mucilage in the form of substituted mannan selected from the group consisting of manno-galactans and gluco-galactans.

U.S. Pat. No. 3,871,892 describes the preparation of pullulan esters by the reaction of pullulan with aliphatic or aromatic fatty acids or their derivatives in the presence of suitable solvents and/or catalysts. The pullulan esters can be shaped by compression molding or extrusion at elevated temperatures or by evaporation of solvents from their solutions to form shaped bodies such as films or coatings.

U.S. Pat. No. 3,873,333 discloses adhesives or pastes prepared by dissolving or dispersing uniformly a pullulan ester and/or ether in water or in a mixture of water and acetone in an amount between 5 percent to 40 percent of the solvent.

U.S. Pat. No. 3,932,192 describes a paper coating material containing pullulan and a pigment.

U.S. Pat. No. 4,257,816 discloses a novel blend of algin, TKP, and guar gum which are useful in commercial gum applications, particularly for the paper-industry, where thickening, suspending, emulsifying, stabilizing, film-forming and gel-forming are needed.

U.S. Pat. No. 3,997,703 discloses a multilayered molded plastic having at least one layer comprising pullulan and at least one layer selected from the group consisting of layers composed of homopolymers and copolymers of olefins and/or vinyl compounds, polyesters, polyamides, celluloses, polyvinylalcohol, rubber hydrochlorides, paper, and aluminum foil.

GB 1,533,301 describes a method of improving the water-resistance of pullulan by the addition of water-soluble dialdehyde polysaccharides to pullulan.

GB 1559 644 also describes a method of improving the water-resistance of pullulan articles. The improved articles are manufactured by means of a process comprising bringing a mixture or shaped composition of a (a) pullulan or a water soluble derivative thereof and (b) polyuronide or a water-soluble salt thereof in contact with an aqueous and/or alcoholic solution of a di- or polyvalent metallic ion.

Although capsules were mentioned or claimed in these patents, their compositions do not have sufficient setting ability or none at all. Consequently, these compositions do not allow an industrial scale hard capsule to be produced, and no attempt has been described to produce pullulan hard capsules by means of conventional dip molding processes.

WO 01/07507 is another reference related to pullulan and owned by the same entity as this case. This reference generally describes pullulan film compositions and setting systems while the current case describes further refinements.

Another problem with conventional pullulan hard capsules is their poor surface gliding performance, which leads to a high opening force of the pre-joint capsules and a high closing force. Indeed, these are two key parameters for a good filling performance on automatic high speed capsule filling equipment. During the filling process, the filling equipment opens, fills and recloses the capsules in an extremely high cadence. High opening or closing force can lead to defects such as unopened, punched capsule ends and consequently to frequent machine stops.

Further problems with conventional capsules are their pour dissolution properties in cold water. A further problem may be that the conventional films or shells do not dissolve without a residue.

The object of the present invention is, therefore, the provision of improved pullulan compositions which overcome the drawbacks of the prior art compositions. This object is solved according to the film forming composition, the container for unit or multiple dosage, the caplets, the capsules, the aqueous solutions, the use of the aqueous solutions for the manufacturing of hard capsules in a dip molding process, manufacturing of hard capsules from aqueous pullulan solutions, and the use of pullulan comprising articles according to the independent claims.

One of the advantages of the present invention is that the pullulan films or containers (e.g. capsules) provide an improved chemical stability, e.g. no cross-linking as described above for gelatin capsules. This is particularly important for the dissolution profile which is not affected during a longer time of storage.

The pullulan compositions provide the further advantage that they are of non-animal origin and that they are compatible with all major excipients.

The present invention provides e.g. a preparation for intratracheobronchial or intranasal administration comprising e.g. a powder preparation or a liquid preparation for intratracheobronchial administration contained in a container, like a capsule, composed of at least pullulan and a setting system. These preparations can be administered through the nose or the mouth.

Further advantageous features, aspects and details of the invention are evident from the dependent claims, the description and the drawings. The claims are to be understood as a first non-limiting approach to define the invention in general terms.

One object of the invention is to provide compositions based on pullulan to improve and adjust the mechanical properties of films for various applications.

The invention provides a film-forming composition comprising pullulan and a setting system.

### SUMMARY OF THE INVENTION

Surprisingly, we found that the addition of a very small amount of a setting system, preferably comprising hydrocolloids acting as a gelling agent, most preferably polysaccharides, confers an appropriate setting ability with cooling to pullulan solution so that the production of hard pullulan capsules can be produced with a conventional dip molding process. One such example of a conventional process may be found in U.S. Pat. No. 2,869,178, which is hereby incorporated by reference as to the description of the process and capsules. Other references of disclosed processes which are hereby incorporated by reference as to the description of the process and capsules include U.S. Pat. Nos. 3,632,700; 3,756,759; 3,794,453; 4,997,359; and 5,769,267.

The addition of a setting system, preferably based on polysaccharides, to pullulan solutions enables the adaptation of specific and desired gelling properties for a selected process (film forming or dip moulding such as the production of hard pullulan capsules by a conventional dipping process). For the production of hard capsules by dip molding process, it is extremely important that the film forming pullulan solution remaining on the mold pins after dipping is prohibited from flowing down the pins. Otherwise the obtained film will not have the desired uniform thickness.

### BRIEF DESCRIPTION OF THE DRAWINGS

FIG. 1 shows dissolution test results of capsules filled with acetaminophen in deionized water at 37° C. (USP XXIII dissolution) from Example 1.

FIG. 2 shows a comparison of the initial disintegration times of gelatin and pullulan capsules as assessed by gamma scintigraphy in fasted volunteers. All capsules disintegrated initially in the stomach.

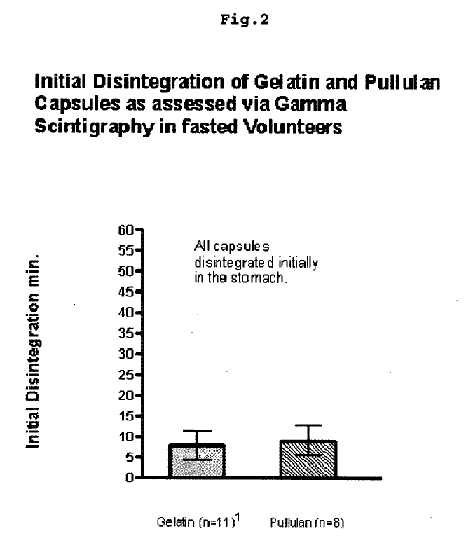

FIG. 3 shows a comparison of the times for complete disintegration of gelatin and pullulan capsules as assessed by gamma scintigraphy in fasted volunteers.

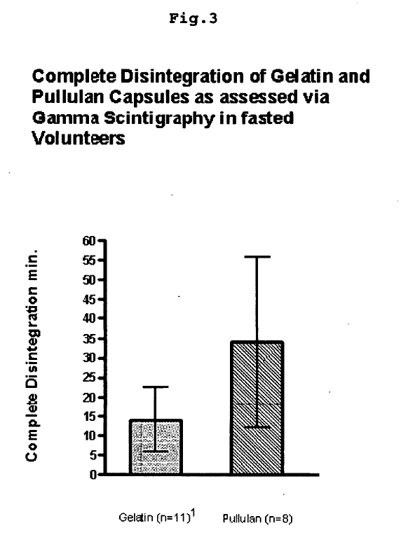

### DETAILED DESCRIPTION OF THE INVENTION AND DESCRIPTION OF THE PREFERRED EMBODIMENTS

In a preferred embodiment, the film forming composition may preferably further contain a cation containing salt, comprising at least one cation. Optionally, the film forming composition may further comprise at least one sequestering agent.

In an aspect of the present invention the film compositions are used for the manufacturing of hard capsules by conventional dip molding process as normally used in the production of conventional hard gelatin capsules.

In an additional aspect of the present invention there are provided aqueous solutions comprising the film forming compositions of the present invention for the manufacture of capsules. The setting system gets the solution to set on the dipped pins, thus assuring a uniform capsule shell thickness. The setting system is preferably composed of a gelling agent, such as said hydrocolloids or polysaccharides, and optionally salt and/or sequestering agent.

The cation containing salt in the composition serves to enhance the setting ability of the gelling agents. Preferably, the salt comprises cations such as K⁺, Li⁺, Na⁺, NH₄⁺, Ca²⁺, or Mg²⁺, etc., for carrageenan K⁺, NH₄⁺ or Ca²⁺ is preferred. The amount of cations is preferably less than 3%, more preferably less than 2% and, most preferred, 0.01 to 1% by weight in the aqueous pullulan solution. The preferred salt concentration in films or capsules is 1.0% to 1.5% by weight. A preferred salt is potassium chloride.

In a further aspect of the present invention there are provided compositions for the use in pharmaceutical, veterinary, food, cosmetic or other products like films for wrapping food, aspics or jellies, preferably for predosed formulations like soft or hard capsules and wherein the pullulan compositions has in aqueous solution a sufficient setting ability.

We found that the addition of a plasticizer in the formulation can improve dramatically the pullulan film flexibility.

In a particular aspect of the present invention there are provided containers for unit or multi dosage forms for agrochemicals, seeds, herbs, foodstuffs like spices, beverages, “instant drinks”, dyestuffs, fertilizers, cosmetics, pharmaceuticals, or flavoring agents produced from the film forming compositions of the present invention. Preferably, such containers are bags, twist-off containers and capsules, capsules, especially pharmaceutical capsules

The capsule halves of the capsules are preferably sealed with one or more layers of the film forming compositions of the present invention. The capsule halves are preferably sealed by means of a liquid fusion process.

Furthermore, the capsules may be formed with two or more non-separable compartments within each capsule for containing different medicaments therein.

With these kind of capsules it is possible to make combinations of two or more medicaments which cannot be combined in a tablet or in a prior art capsule because they tend to react with each other to yield unsuitable products.

With the present invention capsule dosage forms can be provided which can be divided into subunits to be swallowed.

In addition, with the present invention it is possible to provide capsules to be filled with two or more different medicaments.

The capsules of the present invention may preferably release the product they are filled with at low temperatures, preferably at room temperature.

In a further aspect of the present invention there are provided caplets encapsulated in a film forming composition of the present invention.

Compared to gelatin or HPMC, the advantages of pullulan can be mentioned as follows:
Non-animal origin
No chemical modification, totally natural.
Higher product quality consistency by the fermentation process control.
High homogeneity and transparency of films
Very low oxygen permeability. Its capsules are particularly useful for the filling of oxygen sensitive products such as fish and vegetable oils.
Relatively low water content, lower than gelatin.
Low toxicity
No crosslinking
High stability of various properties over storage such as mechanical and dissolution properties.
   Dissolution in cold water
   Dissolution without residues
Better smell and taste masking properties
Improved microbiological quality
Less adhesive forces between pullulan surface and other compounds

The addition of a setting system, preferably based on polysaccharides, to pullulan solutions enables the adaptation of specific and desired gelling properties for the production of hard pullulan capsules by a conventional dipping process. For the production of such capsules it is extremely important that the film forming pullulan solution remaining on the mold pins after dipping is prohibited from flowing down the pins. Otherwise the obtained film will not have the desired uniform thickness.

Consequently the present invention enables that the hard pullulan capsules can be produced with the same equipment used for the production of conventional hard gelatin capsules in the same range of process conditions. Furthermore capsules produced from compositions of the present invention have the same dimensional specifications, allow the use of the existing filling machinery and do not require specific and new equipment for the filling process.

In a preferred embodiment of the present invention, the concentration of pullulan in the dipping aqueous solution is in a range of 10 to 60%, preferably 10 to 50%, more preferably 15 to 40%, and most preferably 10 to 40% by weight.

Although pullulan of various molecular weight is usable, pullulan with a viscosity from 100 cps to 2000 cps at above mentioned concentration and at dipping temperature (40-70° C.) is preferred.

The pullulan without desalting (Japanese food grade) is usable, however the desalted pullulan (Japanese pharmaceutical excipients grade) is preferable for its improved mechanical properties.

Surprisingly, it has been found that the addition of a very small amount of a setting system, preferably consisting of hydrocolloids, most preferably polysaccharides, confers to pullulan solution an appropriate setting ability with the result that hard pullulan capsules can be manufactured by the dip molding process of hard gelatine capsules under conventional process conditions.

The aim of the invention is therefore the provision of compositions based on pullulan for the use in pharmaceutical, veterinary, food, cosmetic or other products like films for wrapping food, aspics or jellies, preferably for containers for predosed formulations like soft or hard capsules and wherein the pullulan compositions have in aqueous solution a sufficient setting ability.

One object of the invention is to provide compositions based on pullulan to improve and adjust the mechanical properties of films for various applications.

It has also been found that it is possible to further improve the film mechanical properties, by combining the pullulan with other hydrosoluble polymers or polysaccharides. The preferable examples are pectin, alginates, polyvinyl alcohol and high molecular weight polyethylene glycol.

Another object of the present invention is the achievement of an adequate setting ability of the pullulan solution for process purpose.

The addition of a setting system, preferably based on polysaccharides, to pullulan solutions enables the adaptation of specific and desired gelling properties for a selected process (film forming or dip molding such as the production of hard pullulan capsules by a conventional dipping process). For the production of hard capsules by dip molding process, it is extremely important that the film forming pullulan solution remaining on the mold pins after dipping is prohibited from flowing down the pins. Otherwise the obtained film will not have the desired uniform thickness.

In preferred embodiments of the present invention the setting system comprises a hydrocolloid or mixtures of hydrocolloids.

Suitable hydrocolloids or mixtures thereof for the present invention, producing synergistic properties, may be selected from the group comprising natural seaweeds, natural seed gums, natural plant exudates, natural fruit extracts, biosynthetic gums, gelatins, biosynthetic processed starch or cellulosic materials. Preferred are the polysaccharides.

In a preferred embodiment of the present invention, the polysaccharides are selected from the group comprising alginates, agar gum, guar gum, locust bean gum (carob), carrageenan, tara gum, gum arabic, ghatti gum, Khaya grandifolia gum, tragacanth gum, karaya gum, pectin, arabian (araban), xanthan, gellan, starch, Konjac mannan, galactomannan, funoran, and other exocellular polysaccharides. Preferred are exocellular polysaccharides.

Preferred exocellular polysaccharides for use in the present invention are selected from the group comprising xanthan, acetan, gellan, welan, rhamsan, furcelleran, succinoglycan, scleroglycan, schizophyllan, tamarind gum, curdlan, and dextran.

In a further preferred embodiment of the present invention the hydrocolloids of the setting system are kappa-carrageenan or gellan gum or combinations like xanthan with locust bean gum or xanthan with konjac mannan.

Among the setting systems mentioned above, the systems of kappa-carrageenan with cations and gellan gum with cations are specifically preferred. They produce high gel strength at low concentrations and have good compatibility with pullulan.

The amount of the setting agent is preferably in the range of 0.01 to 5% by weight and especially preferred to be in the range of 0.03 to 1.0% in the aqueous pullulan solution of the present invention.

The preferred concentration of setting agent in films or capsules is 0.1% to 0.4% by weight. A preferred setting agent is carageenan.

The setting system consists of a hydrocolloid or mixtures of hydrocolloids and may contain in addition cations and/or sequestering agents.

The cations are preferably selected from K⁺, Na⁺, Li⁺, NH₄⁺, Ca⁺⁺ or Mg⁺⁺, for carrageenan, in particular kappa-carrageenan, K⁺, NH₄⁺ or Ca⁺⁺ is preferred. The amount of cations is preferably less than 5%, more preferably 0.01 to 3%, and especially preferred to be in the range of 0.01 to 1% by weight in the aqueous pullulan solution. The preferred salt concentration in films or capsules is 1.0% to 1.5% by weight. A preferred salt is potassium chloride.

In a preferred embodiment of the present invention, the film-forming compositions further comprise one or more sequestering agents.

In a further preferred embodiment of the present invention the sequestering agents are selected from the group comprising ethylenediaminetetraacetic acid, acetic acid, boric acid, citric acid, edetic acid, gluconic acid, lactic acid, phosphoric acid, tartaric acid, or salts thereof, methaphosphates, dihydroxyethylglycine, lecithin or beta cyclodextrin and combinations thereof. Especially preferred is ethylenediaminetetraacetic acid or salts thereof or citric acid or salts thereof. Preferably the amount of sequestering agent is in the range of 0.08% or less by weight in the aqueous pullulan solution. The preferred concentration of sequestering agent in films or capsules is 3.0% by weight or less, more preferably 1.0% by weight or less.

In another preferred embodiment of the present invention, the amount of the sequestering agent is preferably less than 3%, especially 0.01 to 1% by weight of the aqueous dipping solution.

The pullulan compositions of the present invention may in a further preferred embodiment additionally comprise pharmaceutically or food acceptable coloring agents in the range of from 0% to 10% based upon the weight of the film. The coloring agents may be selected from the group comprising azo-, quinophthalone-, triphenylmethane-, xanthene- or indigoid dyes, iron oxides or hydroxides, titanium dioxide or natural dyes or mixtures thereof. Examples are patent blue V, acid brilliant green BS, red 2G, azorubine, ponceau 4R, amaranth, D+C red 33, D+C red 22, D+C red 26, D+C red 28, D+C yellow 10, yellow 2 G, FD+C yellow 5, FD+C yellow 6, FD+C red 3, FD+C red 40, FD+C blue 1, FD+C blue 2, FD+C green 3, brilliant black BN, carbon black, iron oxide black, iron oxide red, iron oxide yellow, titanium dioxide, riboflavin, carotenes, anthocyanines, turmeric, cochineal extract, clorophyllin, canthaxanthin, caramel, or betanin.

The inventive pullulan compositions may in a further preferred embodiment additionally contain at least one pharmaceutically or food acceptable plasticizer or flavoring agent.

It has been found that the addition of a plasticizer in the formulation can improve dramatically the pullulan film flexibility. The plasticizer or mixture of plasticizers is selected from polyethylene glycol, glycerol, sorbitol, sucrose, corn syrup, fructose, dioctyl-sodium sulfosuccinate, triethyl citrate, tributyl citrate, 1,2-propylenglycol, mono-, di- or triacetates of glycerol, or natural gums. Preferred are glycerol, polyethylene glycol, propylene glycol, citrates and their combinations. The amount of plasticizer depends on the final application. For hard film formulations, such as for hard capsules, the plasticizer is contained in an amount of 0 to 20%, preferably 10-20%. A higher content, 20-40%, is preferred for soft film formulations, such as for soft capsules.

The pullulan compositions of the present invention may in a further preferred embodiment additionally comprise pharmaceutically or food acceptable flavoring agents. These may be in the range of from 0% to 10% based upon the weight of the film. Suitable flavoring agents include natural flavors, natural fruit flavors, artificial flavors, artificial fruit flavors, flavor enhancers or mixtures thereof. Natural flavors, artificial flavors or mixtures thereof include, and are not limited to, mint (such as peppermint or spearmint), menthol, cinnamon, vanilla, artificial vanilla, chocolate, artificial chocolate or bubblegum. Natural fruit flavors, artificial fruit flavors or mixtures thereof include, and are not limited to, cherry, grape, orange, strawberry or lemon. Flavor enhancers include, and are not limited to, citric acid. The following flavoring agents or enhancers are only exemplary and include aspartame, autolyzed yeast, corn syrup, disodium guanylate, disodium inosinate, ethyl vanillin, mannitol, monosodium glutamate, potassium glutamate, saccharin, sodium chloride, sorbitol, sucrose, vanilla, xylitol.

Flavoring agents are generally provided as a minor component of the formulation in amounts effective to provide a palatable flavor to the formulation.

In yet another preferred embodiment of the present invention the pullulan containers, such as capsules may be coated with a suitable coating agent like cellulose acetate phthalate, polyvinyl acetate phthalate, methacrylic acid gelatins, hypromellose phthalate, hydroxypropylmethyl cellulose phthalate, hydroxyalkyl methyl cellulose phthalates, hydroxypropyl methylcellulose acetate succinate or mixtures thereof to provide e.g. enteric properties.

In a preferred embodiment of the present invention, the film-forming compositions further comprise one or more surfactants.

The surfactant in the compositions improves the capsule surface properties in such a way that the capsule works well on the conventional automatic high speed capsule filling equipment.

We have surprisingly found that with the addition of a small quantity of selected surfactants of pharmaceutical or food quality, we can improve dramatically the pullulan film surface gliding performance, thereby getting the capsule opening and closing forces into the range required by filling equipment.

Therefore, the present invention provides compositions for hard pullulan capsules with improved surface properties containing pullulan, setting system and surfactant and the aqueous solutions of said film forming compositions for the manufacturing of the capsules.

With these preferably aqueous solutions, we can produce hard pullulan capsules with good filling performance by conventional dip mold process just like hard gelatin capsules.

A further perceived disadvantage of unmodified pullulan capsule film is its adhesive nature or tackiness when touched by hand. The rapid remoisturing properties of pullulan results in a perceived tackiness when holding the capsule film in the hand for 30 seconds or more.

An additional disadvantage is evident on swallowing the capsule film as the film may adhere to the tongue, palet (upper mouth), throat or oesophagus and compare unfavorably with traditional gelatin film capsules. Patient compliance is a major advantage of the traditional hard gelatin capsule and supported by several market studies which cite “ease of swallow” as an important factor in patient preference for the capsule oral dosage form.

In order to solve this perceived disadvantage of unmodified pullulan capsule film, surprisingly it has been found that a surfactant content in the pullulan capsule film provides an acceptable temporary water-repellant surface for handling or swallowing the capsule.

Furtheron a surfactant may be applied externally as a powder or oil in the range of 75 to 500 ppm and more preferably 0.5 to 100 ppm, most preferably 0.5 to 5 ppm.

The pullulan in the compositions is the base material for hard capsule making. Its preferred concentration in the aqueous solutions comprising the surfactant is from 10 to 40%.

The preferred gelling agents for the use with the surfactant are kappa-carrageenan and/or gellan with a concentration in the solutions 0.05-3%.

The surfactant in the compositions is aimed to improve the capsule surface gliding performance, and also the capsule filling performance on filling equipment. The surfactant can be cationic, anionic, non-ionic or amphoteric, and preferably selected from pharmaceutical and food quality such as sodium lauryl sulphate (SLS), dioctyl sodium sulfosuccinate (DSS), benzalkonium chloride, benzethonium chloride, cetrimide (trimethyltetradecyl-ammonium bromide), fatty acid sugar esters, glyceryl monooleate, polyoxyethylene sorbitan fatty acid esters, polyvinyl alcohol, dimethylpolysiloxan, sorbitan esters or lecithin. Its amount based on pullulan is preferably 0.01% to 3%.

It has been found, that while a powder preparation is contained in the conventional various containers like gelatin capsules or blisters and stored, or while it is charged in the administering devices, the powder comes in contact with the container, so that the fine particles of a medicament become adsorbed and adhered to the container surfaces. When administering a medicament into the airway the fine particles adsorbed and adhered to the inner surface of the container will remain in the container. It is a concern of formulators that the emptying of the powder from the container and from the inhaler device may not be reproducible. In a preferred embodiment of the invention a preparation for intratracheobronchial administration comprising e.g. a powder preparation or a liquid intranasal preparation for intratracheobronchial administration contained in a container, like a capsule, composed of at least pullulan and a setting system is provided. Further there is less powder retention of the contained compounds to the container walls since the container walls do not exhibit a high static charge.

In another preferred embodiment according to the invention a preparation for dissolution in cold water comprising a liquid, semi-solid or solid preparation for dissolution in cold water contained in a container, like a capsule, composed of at least pullulan and a setting system is provided. The preparations can be selected from fertilizers, seeds, food additives and or ingredients, like baking powder, flavoring agents, washing powder, instant drinks, beverages, spices etc.

Another embodiment of the invention as a preparation for dissolving without any residues in a liquid, preferably in an aqueous medium, comprising a liquid, semi-solid or solid preparation for dissolving without any residues contained in a container, like a capsule, composed of at least pullulan and a setting system is provided. The preparations can be selected from washing powder, sterilizer for drinking water, and topical microemulsions to be reconstituted in water before application.

The compositions according to the invention can be used for providing packaging of e.g. seeds, micro-chips, dyes, paint etc.

In another preferred embodiment according to the invention a preparation for oral administration comprising a liquid, semi-solid or solid preparation including at least one compound of bad taste contained in a container, like a capsule, composed of at least pullulan and a setting system is provided. It has been found, that the containers according to the invention provide taste masking properties.

The following examples and tests, are not limiting and demonstrate the pullulan capsule production and properties.

Furthermore, the examples demonstrate the hard capsule making, the surface gliding improvement, and the capsule filling improvement.

### EXAMPLE 1

1.0 kg of pullulan (PI-20, Japanese Pharmaceutical Excipients grade) powder is mixed with 10 g of kappa-carrageenan. To 4.0 kg of deionized water under stirring at room temperature is added 20 g of potassium acetate (0.2% by weight in the solution), followed by addition of the above mixture (20% of pullulan and 0.2% of carrageenan in the solution). The powder addition and stirring speeds should be very high in order to avoid the forming of lumps, which take a long time to be dissolved. The solution is heated up to 70° C. under stirring to totally dissolve the carrageenan and pullulan. It is possible to dissolve the components directly at 70° C., but the tendency of pullulan to lump is much higher.

The pullulan solution thus prepared is defoamed under slow stirring and then poured into a dipping dish of a pilot machine of conventional hard gelatin capsule production equipment. While keeping the dipping pullulan solution at 60° C., natural transparent hard pullulan capsules of size 0 were produced according to the conventional process with the same dimensional specifications to the conventional hard gelatin capsules.

### EXAMPLE 2

1.0 kg of pullulan (PI-20) powder is mixed with 6 g of gellan. To 4.0 kg of deionized water under stirring at room temperature is added 20 g of potassium acetate (0.4% by weight in the solution) and 2 g of ethylenediaminetetraacetic acid disodium salt (0.04% in the solution), followed by addition of the above mixture (20% of pullulan and 0.12% of gellan in the solution). The solution is heated up to 75° C. under stirring to dissolve totally the gellan and pullulan.

The pullulan solution thus prepared is defoamed under slow stirring and then poured into a dipping dish of a pilot machine of conventional hard gelatin capsule production equipment. While keeping the dipping pullulan solution at 60° C., natural transparent hard capsules of size 0 were produced according to the conventional process with the same dimensional specifications to the conventional hard gelatin capsules.

Disintegration Test Results:

TABLE 1
Disintegration test results (according to USP
XXIII 1995-<701> Disintegration):

| Capsule | Example 1 | Example 2 |
|---|---|---|
| Capsule emptied time | 3.0 min | 2.0 min |
| Total disintegration time | 10.0 min | 11.8 min |

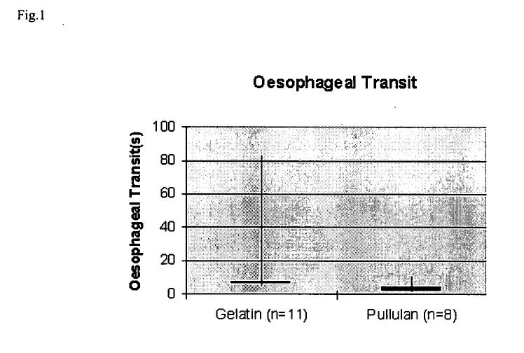

Dissolution test results of capsules filled with acetaminophen in deionized water at 37° C. (USP XXIII dissolution) are represented in FIG. 1.

### EXAMPLE 3

### Pullulan Film Gliding Improvement

In 400 g of demineralized water at room temperature were dispersed under stirring 0.05 g SLS (500 ppm/pullulan), 1 g of kappa-carrageenan (0.2%), 1.25 g of potassium acetate (0.25%) and 100 g of pullulan (20%). The mixture is heated to 70° C. under stirring for complete solubilization and then the stirring is reduced for defoaming. The solution then is used to cast on glass plates of 4 mm thickness to form pullulan films of about 100 μm thickness after drying at room conditions.

The pullulan film gliding performance is evaluated by a test on a slanted plan, a method commonly used by gelatin producers. The method determines the smallest angle of inclination of glass plate to provoke the gliding of a film coated glass plate over another one with films face to face. Consequently, the smaller the gliding angle, the better the film gliding performance.

Repeat the above example with surfactant contents listed in Table 2.

In Table 2, the gliding performance can be evaluated as affected by different surfactants and quantity.

TABLE 2
Pullulan gliding performance (°)

| Surfactant | No | 500 ppm | 1000 ppm | 5000 ppm |
|---|---|---|---|---|
| SLS | 29 | 9 | 5 | 6 |
| Hydrolyzed | | 9 | 9 | 7 |
| deoil lecithin | | | | |
| Polysorbate 20 | | 12 | 12 | |
| Polysorbate 80 | | 10 | 9 | |
| Sorbitan laurate | | 10 | 8 | |
| Sorbitan oleate | | 9 | 7 | |

### EXAMPLE 4

### Pullulan Capsule Production and Performance

In 142 liters of demineralized water at room temperature were dispersed under stirring 20 g hydrolyzed deoil lecithin (500 ppm/pullulan), 363 g kappa-carrageenan (0.2%) and 40 kg pullulan (22%). The mixture is heated up to 70° C. under stirring for total solubilization. 455 g potassium acetate previously dissolved in some water is then added into the solution. A slurry made with 800 g TiO₂, 3 liters hot water and 3 liters so prepared pullulan solution by high shearing is added into the solution in order to produce white opaque capsules. After defoaming, the solution is finally stable at 60° C.

A second identical preparation was made. The two preparations were used to feed a conventional hard gelatin capsule production machine and white opaque hard pullulan capsules were then produced in a similar way to hard gelatin capsules.

As reference, transparent pullulan hard capsules without surfactant in the formulation were produced in the same way as above.

The improvement of hard pullulan capsules by the addition of surfactant is illustrated by the data found in Table 3, and was confirmed by a filling trial on a filling equipment KGF400.

TABLE 3

| | Opening force of pre- | |
|---|---|---|
| Capsule | lock capsule | Closing force |
| Capsule of | 12 g | 6.0 N |
| Example 2 | | |
| Reference | 36 g | 7.6 N |
| capsule | | |

Preferred are hard capsules comprising pullulan in an amount of 85% to 90% by weight, potassium chloride in an amount of 1.0% to 1.5% by weight, carrageenan in an amount of 0.1% to 0.4% by weight, one or more surfactants in an amount of 0.1% to 0.2% by weight and water in an amount of 10% to 15% by weight.

Especially preferred are hard capsules comprising pullulan in an amount of 86.3% by weight, potassium chloride in an amount of 1.32% by weight, carrageenan in an amount of 0.27% by weight, surfactants in an amount of 0.15% by weight and water in an amount of 12% by weight and particularly wherein the surfactants are selected from sugar esters and/or sorbitan monolaurate.

Surprisingly it has been found that hard capsules according to the preferred composition have an improved disintegration behavior.

Oesophageal Transit and In Vivo Disintegration Time

Oesophageal transit and in vivo disintegration time of two-piece capsules made from pullulan have been determined using gamma scintigraphy.

Pullulan capsules according to the preferred compositions were produced using a conventional dip molding process.

The capsule was filled with a placebo formulation containing ¹¹¹In labeled Amberlite® IRP-69 resin. Gamma scintigraphy was used to monitor oesophageal transit and in vivo disintegration properties of the capsules in a group of eight fasted healthy volunteers.

Capsule Composition

| | |
|---|---|
| Base Polymer: | pullulan (86.26% w/w) |
| Setting Agent: | carrageenan (0.27% w/w) |
| Gelling Promoter: | KCl (1.33% w/w) |
| Surfactant: | SE/SML (0.15% w/w |

Capsules (size 2) were manufactured using the conventional dipping process.

Capsule Fill Material

| | |
|---|---|
| Lactose | 148.3 mg |
| Avicel | 45.2 mg |
| Mg Stearate | 1.5 mg |
| | 195.0 mg |

The capsule formulations were radio labeled via the blending of ^{III}In-labeled Amberlite IRP-69 resin into the fill material by using conventional labeling strategies.
Study Design

A single dose investigation was performed in a group of 8 subjects to evaluate the intestinal performance of pullulan and gelatin capsules. Continuous scintigraphic imaging was used to assess oesophageal transit and capsule disintegration properties. The group of 8 subjects was exposed to fasted dosing.

The results obtained for pullulan have been compared with previous values obtained for size 1 gelatin capsules.

Scintigraphic evaluation showed that oesophageal transit time of capsules ranged from 1.3 to 10.8 sec with a mean value of 5 sec. Initial capsule disintegration occurred quickly in the stomach, on average within 9 min post-dose (range x to y). Complete capsule disintegration subsequently occurred on average 34 min post dose and with the exception of one volunteer also took place in the stomach.

The oesophageal transit and in vivo disintegration performance of pullulan capsules in the fasted state compare favorably with previous values obtained with gelatin. Therefore, when a non gelatin capsule is required, two-piece units based on the polysaccharide pullulan can be considered as an alternative choice to gelatin.

1. A preparation for intratracheobronchial administration comprising a container and contents contained in the container, wherein (a) the contents comprise a powder preparation or a liquid preparation for intratracheobronchial administration and (b) the container comprises pullulan and a setting system.

2. A preparation for dissolution in a first liquid without any residue, wherein the preparation comprises a container and contents contained in the container, wherein the contents comprise a second liquid, a semi-solid or a solid preparation contained in the container and the container comprises pullulan and a setting system.

3. A preparation as claimed in claim 2 wherein the first liquid is an aqueous medium.

4. A preparation as claimed in claim 2 wherein the first liquid is cold water.

5. A preparation for oral administration comprising a container and contents contained in the container, wherein (a) the contents are in the form of a liquid, semi-solid or solid and comprise a bad tasting or bad smelling component and (b) the container comprises pullulan and a setting system.

6. A preparation according to any one of claims 1 to 5 wherein the container further comprises one or more additional ingredients selected from the group consisting of sequestering agents, plasticizers, coloring agents, surfactants and flavoring agents.

7. A preparation according to any one of claims 2 to 5 wherein the contents contains at least one additional member selected from the group consisting of fertilizers, seeds, food additives, food ingredients, flavoring agents, vitamins, washing powder, sterilizers for drinking water, and topical microemulsions.

8. A preparation according to any one of claims 1 to 5 wherein the container is a capsule.

9. A preparation according to any one of claims 1 to 5 wherein the capsule is a hard capsule.

10. A preparation according to any one of claims 1 to 5 wherein the capsule is a soft capsule.

11. A hard capsule comprising pullulan in an amount of 85% to 90% by weight, potassium chloride in an amount of 1.0% to 1.5% by weight, carrageenan in an amount of 0.1% to 0.4% by weight, one or more surfactants in an amount of 0.1% to 0.2% by weight and water in an amount of 10% to 15% by weight.

12. A hard capsule according to claim 11 comprising pullulan in an amount of 86.3% by weight, potassium chloride in an amount of 1.32% by weight, carrageenan in an amount of 0.27% by weight, surfactants in an amount of 0.15% by weight and water in an amount of 12% by weight.

13. A hard capsule according to claim 11 or 12, wherein the surfactants are selected from sugar esters, sorbitan monolaurate and mixtures of the foregoing.

14. A hard capsule according to claim 11 or 12, further containing coloring agents in an amount of 0% to 10% by weight.

15. A hard capsule according to claim 11 or 12, having an in vivo dissolution time between 5 and 37 minutes.
